(19) United States
(12) Patent Application Publication
(10) Pub. No.: US 2017/0224675 A1
(43) Pub. Date: Aug. 10, 2017

(54) Treatment of Multiple Sclerosis With Combination of Laquinimod and Dimethyl Fumarate

(71) Applicant: Joel Flaxman Kaye, Netanya (IL)

(72) Inventor: Joel Flaxman Kaye, Netanya (IL)

(73) Assignee: Teva Pharmaceutical Industries, Ltd., Petach-Tikva (IL)

(21) Appl. No.: 15/495,406

(22) Filed: Apr. 24, 2017

Related U.S. Application Data
(63) Continuation of application No. 14/853,826, filed on Sep. 14, 2015, now abandoned.
Application No. 14/853,826 is a continuation of application No. 14/853,826, filed on Sep. 14, 2015, now abandoned.
Application No. 14/853,826 is a continuation of application No. 14/589,684, filed on Jan. 5, 2015, now abandoned.
Application No. 14/589,684 is a continuation of application No. 13/800,047, filed on Mar. 13, 2013, now abandoned.
(60) Provisional application No. 61/616,337, filed on Mar. 27, 2012.

(51) Int. Cl. A61K 31/4704; A61K 31/225; A61K 45/06

(52) U.S. Cl. CPC A61K 31/4704; A61K 45/06; A61K 31/225

(57) ABSTRACT

This invention provides a method of treating a subject afflicted with multiple sclerosis or presenting a clinically isolated syndrome comprising administering to the subject laquinimod as an add-on therapy to or in combination with DMF. This invention also provides a package comprising laquinimod and DMF for treating a subject afflicted with multiple sclerosis or presenting a clinically isolated syndrome. This invention also provides laquinimod for use as an add-on therapy or in combination with DMF in treating a subject afflicted with multiple sclerosis or presenting a clinically isolated syndrome. This invention also provides a pharmaceutical composition comprising laquinimod and DMF for use in treating a subject afflicted with multiple sclerosis or presenting a clinically isolated syndrome. This invention further provides use of laquinimod and DMF in the preparation of a combination for treating a subject afflicted with multiple sclerosis or presenting a clinically isolated syndrome.

This application claims benefit of U.S. Provisional Application No. 61/616,337, filed Mar. 27, 2012, the entire content of which is hereby incorporated by reference herein.

Throughout this application, various publications are referred to by first author and year of publication. Full citations for these publications are presented in a References section immediately before the claims. Disclosures of the documents and publications referred to herein are hereby incorporated in their entireties by reference into this application.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care (EMEA Guideline, 2006).

It is generally assumed that MS is mediated by some kind of autoimmune process possibly triggered by infection and superimposed upon a genetic predisposition. It is a chronic inflammatory condition that damages the myelin of the Central Nervous System (CNS). The pathogenesis of MS is characterized by the infiltration of autoreactive T-cells from the circulation directed against myelin antigens into the CNS (Bjartmar, 2002). In addition to the inflammatory phase in MS, axonal loss occurs early in the course of the disease and can be extensive over time, leading to the subsequent development of progressive, permanent, neurologic impairment and, frequently, severe disability (Neuhaus, 2003). Symptoms associated with the disease include fatigue, spasticity, ataxia, weakness, bladder and bowel disturbances, sexual dysfunction, pain, tremor, paroxysmal manifestations, visual impairment, psychological problems and cognitive dysfunction (EMEA Guideline, 2006).

MS disease activity can be monitored by cranial scans, including magnetic resonance imaging (MRI) of the brain, accumulation of disability, as well as rate and severity of relapses. The diagnosis of clinically definite MS as determined by the Poser criteria (Poser, 1983) requires at least two neurological events suggesting demyelination in the CNS separated in time and in location. A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite multiple sclerosis (CDMS). Over 80 percent of patients with a CIS and MRI lesion go on to develop MS, while approximately 20 percent have a self-limited process (Brex, 2002; Frohman, 2003).

Various MS disease stages and/or types are described in Multiple Sclerosis Therapeutics (Duntiz, 1999). Among them, relapsing remitting multiple sclerosis (RRMS) is the most common form at the time of initial diagnosis. Many subjects with RRMS have an initial relapsing-remitting course for 5-15 years, which then advances into the secondary progressive MS (SPMS) disease course. Relapses result from inflammation and demyelination, whereas restoration of nerve conduction and remission is accompanied by resolution of inflammation, redistribution of sodium channels on demyelinated axons and remyelination (Neuhaus, 2003; Noseworthy, 2000).

In April 2001, an international panel in association with the National MS Society of America recommended diagnostic criteria for multiple sclerosis. These criteria became known as the McDonald Criteria. The McDonald Criteria make use of MRI techniques and are intended to replace the Poser Criteria and the older Schumacher Criteria (McDonald, 2001). The McDonald Criteria was revised in March 2005 by an international panel (Polman, 2005) and updated again in 2010 (Polman, 2010).

Intervention with disease-modifying therapy at relapsing stages of MS is suggested to reduce and/or prevent accumulating neurodegeneration (Hohlfeld, 2000; De Stefano, 1999). There are currently a number of disease-modifying medications approved for use in relapsing MS (RMS), which includes RRMS and SPMS (The Disease Modifying Drug Brochure, 2006). These include interferon beta 1-a (Avonex® and Rebif®), interferon beta 1-b (Betaseron®), glatiramer acetate (Copaxone®), mitoxantrone (Novantrone®), natalizumab (Tysabri®) and Fingolimod (Gilenya®). Most of them are believed to act as immunomodulators. Mitoxantrone and natalizumab are believed to act as immunesuppressants. However, the mechanisms of action of each have been only partly elucidated. Immunosuppressants or cytotoxic agents are used in some subjects after failure of conventional therapies. However, the relationship between changes of the immune response induced by these agents and the clinical efficacy in MS is far from settled (EMEA Guideline, 2006).

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

Panaclar®, DMF, BG-12, FAG-201, Dimethyl Fumarate, Dimethyl (E)-but-2-Enedioate

BG-12 is an FAE (fumaric acid ester), an oral formulation of DMF (dimethyl fumarate) with known anti-inflammatory and neuroprotective effects. FAE's were first considered for use as treatment for psoriasis, a Th1-mediated disease, due to anti-proliferative effects on lymphocytes (Stoof et al., 2001; Mrowietz and Asadullah, 2005). Fumaderm, a FAE, has been approved for psoriasis in Europe for over 15 years. Subsequent studies showed that DMF reduces inflammatory gene expression, including that of pro-inflammatory cytokines and chemokines, and increases anti-inflammatory expression (Stoof et al., 2001; Loewe et al., 2002; Seidel et al., 2009)—effects likely to contribute to its anti-psoriasis efficacy. These findings have led to increased interest for using DMF in other auto-immune or inflammatory diseases, including MS (Kappos et al., 2008; Moharregh-Khiabani et al., 2009). In animal studies, DMF reduced glial inflammation during MOG (myelin oligodendrocyte glycoprotein) peptide induced EAE (experimental autoimmune encephalomyelitis) and increased plasma levels of IL-10 (interleukin-10; Schilling et al., 2006). A Phase 2B trial of DMF in RRMS (relapsing remitting MS) patients showed significant decreases in new gadolinium enhancing lesions, T1 and T2 lesions, and a non-significant decrease in the annualized relapse rate (Kappos et al., 2008).

The mechanisms of the action of DMF are not fully known. DMF can suppress NF-κB (nuclear factor κB)-dependent transcription (Stoof et al., 2001; Gerdes et al., 2007), thus accounting for some of its anti-inflammatory effects. DMF can also activate the Nrf2 (nuclear factor-erythroid 2 p45 subunit-related factor 2) pathway (Lukashev et al., 2007; Kappos et al., 2008), which induces the transcription of various genes, including anti-oxidative ones, reduces oxidative neuronal death and helps maintain myelin integrity. DMF induces detoxification enzymes in astrocytes and microglial cells (Wierinckx et al., 2005). As a consequence, DMF can modulate GSH levels in cells leading to cytotoxic or protective effects (Dethlefsen et al., 1988; Spencer et al., 1990), including in primary astrocytes (Schmidt and Dringen, 2010). The anti-inflammatory effects of DMF have been shown, in some cases, to involve induction of HO-1 (haem oxygenase 1) also termed HSP32 (heat-shock protein 32) (Lehmann et al., 2007), which occurs following GSH depletion. HO-1 can suppress a variety of inflammatory responses (Horikawa et al., 2002), as well as confer protection against oxidative stress (Min et al., 2006).

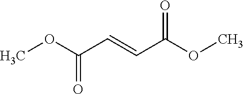

IUPAC Name: Dimethyl (E)-Butenedioate

Laquinimod

Laquinimod is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described, for example, in U.S. Pat. No. 6,077,851. The mechanism of action of laquinimod is not fully understood. Animal studies show it causes a Th1 (T helper 1 cell, which produces pro-inflammatory cytokines) to Th2 (T helper 2 cell, which produces anti-inflammatory cytokines) shift with an anti-inflammatory profile (Yang, 2004; Bruck, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runström, 2006; Bruck, 2011).

Combination Therapy

The administration of two drugs to treat a given condition, such as multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of GA and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of combination therapy of two drugs, in particular laquinimod and DMF, cannot be predicted until the results of formal combination studies are available.

### BRIEF DESCRIPTION OF THE DRAWINGS

FIG. 1 is a graphical representation of the experimental results from Example 1B.

### SUMMARY OF THE INVENTION

This invention provides a method of treating a subject afflicted with a form of multiple sclerosis (MS) or presenting a clinically isolated syndrome (CIS) comprising periodically administering to the subject an amount of laquinimod or pharmaceutically acceptable salt thereof, and an amount of dimethyl fumarate (DMF) or pharmaceutically acceptable salt thereof, wherein the amounts when taken together are effective to treat the subject.

This invention also provides a package comprising: a) a first pharmaceutical composition comprising an amount of laquinimod or pharmaceutically acceptable salt thereof and a pharmaceutically acceptable carrier; b) a second pharmaceutical composition comprising an amount of DMF or pharmaceutically acceptable salt thereof and a pharmaceutically acceptable carrier; and c) instruction for use for the first and the second pharmaceutical composition together to treat a subject afflicted with MS or presenting a clinically isolated syndrome.

This invention also provides laquinimod or pharmaceutically acceptable salt thereof for use as an add-on therapy or in combination with DMF or pharmaceutically acceptable salt thereof in treating a subject afflicted with multiple sclerosis or presenting a clinically isolated syndrome.

This invention also provides a pharmaceutical composition comprising an amount of laquinimod or pharmaceutically acceptable salt thereof and an amount of DMF or pharmaceutically acceptable salt thereof, and at least one pharmaceutical acceptable carrier.

This invention also provides use of: a) an amount of laquinimod or pharmaceutically acceptable salt thereof; and b) an amount of DMF or pharmaceutically acceptable salt thereof in the preparation of a combination for treating a subject afflicted with multiple sclerosis or presenting a clinically isolated syndrome wherein the laquinimod or pharmaceutically acceptable salt thereof and the DMF or pharmaceutically acceptable salt thereof are administered simultaneously or contemporaneously.

This invention also provides a pharmaceutical composition comprising an amount of laquinimod for use in treating a subject afflicted with MS or presenting a clinically isolated syndrome, in combination with an amount of DMF, by periodically administering to the subject the pharmaceutical composition and the amount of DMF.

This invention also provides a pharmaceutical composition comprising an amount of DMF for use treating a subject afflicted with MS or presenting a clinically isolated syndrome, in combination with an amount of laquinimod, by periodically administering to the subject the pharmaceutical composition and the amount of laquinimod.

This invention also provides laquinimod or pharmaceutically acceptable salt thereof and DMF or pharmaceutically acceptable salt thereof for the treatment of a subject afflicted with MS or presenting a clinically isolated syndrome, wherein the laquinimod and the DMF are administered simultaneously, separately or sequentially.

This invention also provides a product containing an amount of laquinimod and an amount of DMF for simultaneous, separate or sequential use in treating a subject afflicted with MS or presenting a clinically isolated syndrome.

### DETAILED DESCRIPTION OF THE INVENTION

This invention provides a method of treating a subject afflicted with multiple sclerosis (MS) or presenting a clinically isolated syndrome (CIS) comprising periodically administering to the subject an amount of laquinimod or a pharmaceutically acceptable salt thereof, and an amount of DMF or a pharmaceutically acceptable salt thereof, wherein the amounts when taken together are effective to treat the subject. In an embodiment, the amount of laquinimod or pharmaceutically acceptable salt thereof and the amount of DMF or pharmaceutically acceptable salt thereof when administered together is more effective to treat the subject than when each agent at the same amount is administered alone.

In one embodiment, the pharmaceutically acceptable salt of laquinimod is administered. In another embodiment, the salt is laquinimod sodium.

In one embodiment, the laquinimod is administered via oral administration. In another embodiment, the laquinimod is administered daily.

In one embodiment, the amount of laquinimod administered is 0.0005-10 mg/kg (mg of drug per kg of body weight of subject) per day. In another embodiment, the amount of laquinimod administered is 0.01 mg/kg per day. In another embodiment, the amount of laquinimod administered is 0.005 mg/kg per day. In another embodiment, the amount of laquinimod is 5 mg/kg per day. In another embodiment, the amount of laquinimod is 10 mg/kg per day.

In another embodiment, the amount of laquinimod is 25 mg/kg per day. In yet another embodiment, the amount of laquinimod is about the above-mentioned amounts.

In one embodiment, the amount of laquinimod administered is 0.03-600 mg/day. In another embodiment, the amount of laquinimod is 0.1-120.0 mg/day. In another embodiment, the amount of laquinimod is 0.1-40.0 mg/day. In another embodiment, the amount of laquinimod is 0.1-2.5 mg/day. In another embodiment, the amount of laquinimod is 0.25-2.0 mg/day. In another embodiment, the amount of laquinimod is 0.5-1.2 mg/day. In yet another embodiment, the amount of laquinimod is about the above-mentioned amounts.

In one embodiment, the amount of laquinimod is 2.0 mg/day. In another embodiment, the amount of laquinimod is 1.5 mg/day. In another embodiment, the amount of laquinimod is 1.2 mg/day. In another embodiment, the amount of laquinimod is less than 1.2 mg/day. In another embodiment, the amount laquinimod is 1.0 mg/day. In another embodiment, the amount of laquinimod administered is 0.6 mg/day. In another embodiment, the amount of laquinimod administered is less than 0.6 mg/day. In another embodiment, the amount laquinimod administered is 0.5 mg/day. In another embodiment, the amount of laquinimod administered is 0.3 mg/day. In another embodiment, the amount laquinimod is 0.25 mg/day. In yet another embodiment, the amount of laquinimod is about the above-mentioned amounts.

In one embodiment, the DMF is administered via oral administration. In another embodiment, the DMF is administered daily.

In one embodiment, the amount of DMF administered is 0.2-120 mg/kg (mg of drug per kg of body weight of subject) per day. In another embodiment, the amount of DMF administered is 12 mg/kg per day. In another embodiment, the amount of DMF administered is mg/kg per day. In another embodiment, the amount of DMF administered is 6 mg/kg per day. In another embodiment, the amount of DMF administered is 4 mg/kg per day. In another embodiment, the amount of DMF administered is 2 mg/kg per day. In another embodiment, the amount of DMF administered is 0.005 mg/kg per day. In yet another embodiment, the amount of DMF is about the above-mentioned amounts.

In one embodiment, the amount of DMF administered is 12 mg/day to 7200 mg/day. In another embodiment, the amount of DMF administered is 120 mg/day to 720 mg/day. In another embodiment, the amount of DMF administered is 720 mg/day. In another embodiment, the amount of DMF administered is less than 720 mg/day. In another embodiment, the amount of DMF administered is 480 mg/day. In another embodiment, the amount of DMF administered is less than 480 mg/day. In another embodiment, the amount of DMF administered is 360 mg/day. In another embodiment, the amount of DMF administered is less than 360 mg/day. In another embodiment, the amount of DMF administered is 240 mg/day. In another embodiment, the amount of DMF administered is less than 240 mg/day. In another embodiment, the amount of DMF administered is 120 mg/day. In another embodiment, the amount of DMF administered is less than 120 mg/day. In yet another embodiment, the amount of DMF is about the above-mentioned amounts.

In an embodiment, the DMF is administered once daily. In another embodiment, the DMF is administered twice daily. In another embodiment, the DMF is administered three times daily.

In one embodiment, the amount of laquinimod or pharmaceutically acceptable salt thereof and the amount of DMF or pharmaceutically acceptable salt thereof when taken together is effective to alleviate a symptom of multiple sclerosis in the subject. In another embodiment, the symptom is a MRI-monitored multiple sclerosis disease activity, relapse rate, accumulation of physical disability, frequency of relapses, frequency of clinical exacerbation, brain atrophy, risk for confirmed progression, or time to confirmed disease progression.

In one embodiment, the accumulation of physical disability is measured by the subject's Kurtzke Expanded Disability Status Scale (EDSS) score. In another embodiment, the accumulation of physical disability is assessed by the time to confirmed disease progression as measured by Kurtzke Expanded Disability Status Scale (EDSS) score. In another embodiment, the subject had an EDSS score of 0-5.5 prior to administration of laquinimod. In another embodiment, the subject had an EDSS score of 5.5 or greater prior to administration of laquinimod. In another embodiment, confirmed disease progression is a 1 point increase of the EDSS score. In another embodiment, confirmed disease progression is a 0.5 point increase of the EDSS score.

In one embodiment, time to confirmed disease progression is increased by at least 30%, compared to a patient not receiving the laquinimod treatment. In another embodiment, time to confirmed disease progression is increased by 20-60%, compared to a patient not receiving the laquinimod treatment. In another embodiment, time to confirmed disease progression is increased by 30-50%, compared to a patient not receiving the laquinimod treatment. In another embodiment, time to confirmed disease progression is increased by at least 50%, compared to a patient not receiving the laquinimod treatment.

In one embodiment, the administration of laquinimod substantially precedes the administration of DMF. In another embodiment, the administration of DMF substantially precedes the administration of laquinimod.

In one embodiment, the subject is receiving laquinimod therapy prior to initiating DMF therapy. In another embodiment, the subject is receiving laquinimod therapy for at least 24 weeks prior to initiating DMF therapy. In another embodiment, the subject is receiving laquinimod therapy for at least 28 weeks prior to initiating DMF therapy. In another embodiment, the subject is receiving laquinimod therapy for at least 48 weeks prior to initiating DMF therapy. In yet another embodiment, the subject is receiving laquinimod therapy for at least 52 weeks prior to initiating DMF therapy.

In one embodiment, the subject is receiving DMF therapy prior to initiating laquinimod therapy. In another embodiment, the subject is receiving DMF therapy for at least 24 weeks prior to initiating laquinimod therapy. In another embodiment, the subject is receiving DMF therapy for at least 28 weeks prior to initiating laquinimod therapy. In another embodiment, the subject is receiving DMF therapy for at least 48 weeks prior to initiating laquinimod therapy. In yet another embodiment, the subject is receiving DMF therapy for at least 52 weeks prior to initiating laquinimod therapy.

In one embodiment, the method further comprises administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

In one embodiment, the periodic administration of laquinimod or pharmaceutically acceptable salt thereof and DMF continues for more than 30 days. In another embodiment, the periodic administration of laquinimod or pharmaceutically acceptable salt thereof and DMF continues for more than 42 days. In yet another embodiment, the periodic administration of laquinimod or pharmaceutically acceptable salt thereof and DMF continues for 6 months or more.

In one embodiment, the administration of laquinimod or pharmaceutically acceptable salt thereof and DMF or pharmaceutically acceptable salt thereof inhibits a symptom of MS, e.g., relapsing multiple sclerosis by at least 30%. In another embodiment, the administration of laquinimod or pharmaceutically acceptable salt thereof and DMF or pharmaceutically acceptable salt thereof inhibits the symptom by at least 50%. In another embodiment, the administration of laquinimod or pharmaceutically acceptable salt thereof and DMF or pharmaceutically acceptable salt thereof inhibits the symptom by more than 100%. In another embodiment, the administration of laquinimod or pharmaceutically acceptable salt thereof and DMF or pharmaceutically acceptable salt thereof inhibits the symptom by more than 300%. In another embodiment, the administration of laquinimod or pharmaceutically acceptable salt thereof and DMF or pharmaceutically acceptable salt thereof inhibits the symptom by more than 1000%.

In one embodiment, each of the amount of laquinimod or pharmaceutically acceptable salt thereof when taken alone, and the amount of DMF or pharmaceutically acceptable salt thereof when taken alone is effective to treat the subject. In another embodiment, either the amount of laquinimod or pharmaceutically acceptable salt thereof when taken alone, the amount of DMF or pharmaceutically acceptable salt thereof when taken alone, or each such amount when taken alone is not effective to treat the subject. In yet another embodiment, the subject is a human patient.

This invention also provides a package comprising: a) a first pharmaceutical composition comprising an amount of laquinimod or pharmaceutically acceptable salt thereof and a pharmaceutically acceptable carrier; b) a second pharmaceutical composition comprising an amount of DMF or pharmaceutically acceptable salt thereof and a pharmaceutically acceptable carrier; and c) instructions for use of the first and the second pharmaceutical compositions together to treat a subject afflicted with multiple sclerosis or presenting a clinically isolated syndrome. In an embodiment, the package is for use in treating a subject afflicted with MS or presenting a clinically isolated syndrome.

This invention also provides laquinimod or pharmaceutically acceptable salt thereof for use as an add-on therapy or in combination with DMF or pharmaceutically acceptable salt thereof in treating a subject afflicted with multiple sclerosis or presenting a clinically isolated syndrome.

This invention also provides a pharmaceutical composition comprising an amount of laquinimod or pharmaceutically acceptable salt thereof, an amount of DMF or pharmaceutically acceptable salt thereof, and at least one pharmaceutically acceptable carrier. In an embodiment, the pharmaceutical composition is for use in treating a subject afflicted with MS or presenting a clinically isolated syndrome.

This invention also provides a pharmaceutical composition comprising an amount of laquinimod or pharmaceutically acceptable salt thereof and an amount of DMF or pharmaceutically acceptable salt thereof for use in treating a subject afflicted with multiple sclerosis or presenting a clinically isolated syndrome, wherein the laquinimod or pharmaceutically acceptable salt thereof and the DMF or pharmaceutically acceptable salt thereof are administered simultaneously or contemporaneously.

In one embodiment, the pharmaceutically acceptable salt of laquinimod is laquinimod sodium.

In one embodiment, the amount of laquinimod in the composition is 0.03-600 mg. In another embodiment, the amount of laquinimod is 0.1-120.0 mg. In another embodiment, the amount of laquinimod is 0.1-40.0 mg. In another embodiment, the amount of laquinimod is 0.1-2.5 mg. In another embodiment, the amount of laquinimod is 0.25-2.0 mg. In another embodiment, the amount of laquinimod is 0.5-1.2 mg. In yet another embodiment, the amount of laquinimod is about the above-mentioned amounts.

In an embodiment, the amount of laquinimod is 0.25 mg. In another embodiment, the amount of laquinimod is 0.5 mg. In another embodiment, the amount of laquinimod is 1.0 mg. In another embodiment, the amount of laquinimod is 1.5 mg. In another embodiment, the amount of laquinimod is 2.0 mg. In another embodiment, the amount of laquinimod is 1.2 mg. In another embodiment, the amount of laquinimod is less than 1.2 mg. In another embodiment, the amount of laquinimod in the composition is 0.6 mg. In another embodiment, the amount of laquinimod in the composition is less than 0.6 mg. In another embodiment, the amount of laquinimod in the composition is 0.3 mg. In yet another embodiment, the amount of laquinimod is about the above-mentioned amounts.

In one embodiment, the amount of DMF in the composition is 12 mg to 7200 mg. In another embodiment, the amount of DMF in the composition is 720 mg. In another embodiment, the amount of DMF in the composition is less than 720 mg. In another embodiment, the amount of DMF in the composition is 480 mg. In another embodiment, the amount of DMF in the composition is less than 480 mg. In another embodiment, the amount of DMF in the composition is 360 mg. In another embodiment, the amount of DMF in the composition is less than 360 mg. In another embodiment, the amount of laquinimod in the composition is 240 mg. In another embodiment, the amount of laquinimod in the composition is less than 240 mg. In another embodiment, the amount of laquinimod in the composition is 120 mg. In another embodiment, the amount of laquinimod in the composition is less than 120 mg/day. In yet another embodiment, the amount of DMF is about the above-mentioned amounts.

In an embodiment, the DMF is formulated for administration once daily. In another embodiment, the DMF is formulated for administration twice daily. In another embodiment, the DMF is formulated for administration three times daily.

This invention also provides use of: a) an amount of laquinimod or pharmaceutically acceptable salt thereof; and b) an amount of

DMF or pharmaceutically acceptable salt thereof in the preparation of a combination for treating a subject afflicted with multiple sclerosis or presenting a clinically isolated syndrome wherein the amount of laquinimod or pharmaceutically acceptable salt thereof and the amount of DMF or pharmaceutically acceptable salt thereof are administered simultaneously or contemporaneously.

This invention also provides a pharmaceutical composition comprising an amount of laquinimod for use in treating a subject afflicted with MS or presenting a clinically isolated syndrome, in combination with an amount of DMF, by periodically administering to the subject the pharmaceutical composition and the amount of DMF.

This invention also provides a pharmaceutical composition comprising an amount of DMF for use treating a subject afflicted with MS or presenting a clinically isolated syndrome, in combination with an amount of laquinimod, by periodically administering to the subject the pharmaceutical composition and the amount of laquinimod.

This invention also provides laquinimod or pharmaceutically acceptable salt thereof and DMF or pharmaceutically acceptable salt thereof for the treatment of a subject afflicted with MS or presenting a clinically isolated syndrome, wherein the laquinimod and the DMF are administered simultaneously, separately or sequentially.

This invention also provides a product containing an amount of laquinimod and an amount of DMF for simultaneous, separate or sequential use in treating a subject afflicted with MS or presenting a clinically isolated syndrome.

In one embodiment of any of above-mentioned methods, pharmaceutical compositions, packages, products and uses, the multiple sclerosis is relapsing multiple sclerosis. In another embodiment, the relapsing multiple sclerosis is relapsing-remitting multiple sclerosis.

For the foregoing embodiments, each embodiment disclosed herein is contemplated as being applicable to each of the other disclosed embodiments. The elements recited in the method embodiments can be used in the pharmaceutical composition, package, product and use embodiments described herein and vice versa.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT

International Application Publication No. WO 2005/074899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248.

General techniques and compositions for making dosage forms useful in the present invention are described-in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds).; Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is a method for treating a subject, e.g., human patient, afflicted with multiple sclerosis, e.g., relapsing multiple sclerosis or presenting CIS using laquinimod with DMF which provides a more efficacious treatment than each agent alone. The use of laquinimod for multiple sclerosis had been previously suggested in, e.g., U.S. Pat. No. 6,077,851. However, the inventors have surprisingly found that the combination of laquinimod and DMF is particularly effective for the treatment of relapsing multiple sclerosis as compared to each agent alone.

### Terms

As used herein, and unless stated otherwise, each of the following terms shall have the definition set forth below.

As used herein, “laquinimod” means laquinimod acid or a pharmaceutically acceptable salt thereof.

As used herein, “dimethyl fumarate” or “DMF”, unless otherwise, specified means dimethyl fumarate or a pharmaceutically acceptable salt thereof.

A “salt thereof” is a salt of the instant compounds which have been modified by making acid or base salts of the compounds. The term “pharmaceutically acceptable salt” in this respect, refers to the relatively non-toxic, inorganic and organic acid or base addition salts of compounds of the present invention. For example, one means of preparing such a salt is by treating a compound of the present invention with an inorganic base.

As used herein, an “amount” or “dose” of laquinimod as measured in milligrams refers to the milligrams of laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion. Similarly, “amount” or “dose” of DMF as measured in milligrams refers to the milligrams of DMF present in a preparation, regardless of the form of the preparation.

As used herein, “about” in the context of a numerical value or range means±10% of the numerical value or range recited or claimed.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the laquinimod and the DMF. In this case, the combination may be the admixture or separate containers of the laquinimod and the DMF that are combined just prior to administration. Contemporaneous administration refers to the separate administration of the laquinimod and the DMF at the same time, or at times sufficiently close together that a synergistic activity or an activity that is additive or more than additive relative to the activity of either the laquinimod or the DMF alone is observed.

“Administration” means the giving of, dispensing of, or application of medicines, drugs, or remedies to a subject to relieve or cure a pathological condition. Oral administration is one way of administering the instant compounds to the subject.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding laquinimod therapy to a patient already receiving DMF therapy.

As used herein, “effective” when referring to an amount of laquinimod and/or DMF refers to the quantity of laquinimod and/or DMF that is sufficient to yield a desired therapeutic response without undue adverse side effects (such as toxicity, irritation, or allergic response) commensurate with a reasonable benefit/risk ratio when used in the manner of this invention.

“Treating” as used herein encompasses, e.g., inducing inhibition, regression, or stasis of a disease or disorder, e.g., MS or RMS, or alleviating, lessening, suppressing, inhibiting, reducing the severity of, eliminating or substantially eliminating, or ameliorating a symptom of the disease or disorder. “Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

“Inhibition” of disease progression or disease complication in a subject means preventing or reducing the disease progression and/or disease complication in the subject.

A “symptom” associated with MS or RMS includes any clinical or laboratory manifestation associated with MS or RMS and is not limited to what the subject can feel or observe.

As used herein, “a subject afflicted with multiple sclerosis” or “a subject afflicted with MS” means a subject who was been clinically diagnosed to have a form of multiple sclerosis.

As used herein, “a subject afflicted with relapsing multiple sclerosis” means a subject who was been clinically diagnosed to have relapsing multiple sclerosis (RMS) which includes relapsing-remitting multiple sclerosis (RRMS) and Secondary Progressive multiple sclerosis (RRMS).

“Relapse Rate” is the number of confirmed relapses per unit time. “Annualized relapse rate” is the mean value of the number of confirmed relapses of each patient multiplied by 365 and divided by the number of days that patient is on the study drug.

“Expanded Disability Status Scale” or “EDSS” is a rating system that is frequently used for classifying and standardizing the condition of people with multiple sclerosis. The score ranges from 0.0 representing a normal neurological exam to 10.0 representing death due to MS. The score is based upon neurological testing and examination of functional systems (FS), which are areas of the central nervous system which control bodily functions. The functional systems are: Pyramidal (ability to walk), Cerebellar (coordination), Brain stem (speech and swallowing), Sensory (touch and pain), Bowel and bladder functions, Visual, Mental, and Other (includes any other neurological findings due to MS) (Kurtzke J F, 1983).

A “confirmed progression” of EDSS, or “confirmed disease progression” as measured by EDSS score is defined as a 1 point increase from baseline EDSS if baseline EDSS was between 0 and 5.0, or a 0.5 point increase if baseline EDSS was 5.5. In order to be considered a confirmed progression, the change (either 1 point or 0.5 points) must be sustained for at least 3 months. In addition, confirmation of progression cannot be made during a relapse.

“Adverse event” or “AE” means any untoward medical occurrence in a clinical trial subject administered a medicinal product and which does not have a causal relationship with the treatment. An adverse event can therefore be any unfavorable and unintended sign including an abnormal laboratory finding, symptom, or diseases temporally associated with the use of an investigational medicinal product, whether or not considered related to the investigational medicinal product.

“Gd-enhancing lesion” refers to lesions that result from a breakdown of the blood-brain barrier, which appear in contrast studies using gandolinium contrast agents. Gandolinium enhancement provides information as to the age of a lesion, as

Gd-enhancing lesions typically occur within a six week period of lesion formation.

“Magnetization Transfer Imaging” or “MTI” is based on the magnetization interaction (through dipolar and/or chemical exchange) between bulk water protons and macromolecular protons.

By applying an off resonance radio frequency pulse to the macromolecular protons, the saturation of these protons is then transferred to the bulk water protons. The result is a decrease in signal (the net magnetization of visible protons is reduced), depending on the magnitude of MT between tissue macromolecules and bulk water. “MT” or “Magnetization Transfer” refers to the transfer of longitudinal magnetization from the hydrogen nuclei of water that have restricted motion to the hydrogen nuclei of water that moves with many degrees of freedom. With MTI, the presence or absence of macromolecules (e.g. in membranes or brain tissue) can be seen (Mehta, 1996; Grossman, 1994).

“Magnetization Resonance Spectroscopy” or “MRS” is a specialized technique associated with magnetic resonance imaging (MRI). MRS is used to measure the levels of different metabolites in body tissues. The MR signal produces a spectrum of resonances that correspond to different molecular arrangements of the isotope being “excited”. This signature is used to diagnose certain metabolic disorders, especially those affecting the brain, (Rosen, 2007) as well as to provide information on tumor metabolism (Golder, 2007).

“T1-weighted MRI image” refers to an MR-image that emphasizes T1 contrast by which lesions may be visualized. Abnormal areas in a T1-weighted MRI image are “hypointense” and appear as dark spots. These spots are generally older lesions.

“T2-weighted MRI image” refers to an MR-image that emphasizes T2 contrast by which lesions may be visualized. T2 lesions represent new inflammatory activity.

A “patient at risk of developing MS” (i.e. clinically definite MS) as used herein is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R-alpha and IL2R-alpha), and immunological components (viral infection such as by Epstein-Barr virus, high avidity CD4⁺ T cells, CD8⁺ T cells, anti-NF-L, anti-CSF 114(Glc)).

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “5-10%” includes 5.0%, 5.1%, 5.2%, 5.3%, 5.4% etc. up to 10.0%.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

Since the mechanisms of action of laquinimod and DMF have not been fully elucidated, the effect of the combined therapy cannot be predicted and must be evaluated experimentally.

### Example 1A: Assessment of Efficacy of Laquinimod Alone or in Combination with DMF in MOG-Induced EAE

In this experiment, MOG-induced EAE Mice are treated with two doses of laquinimod (0.06 and 0.12 mg/kg) alone or with add on DMF (25 or 50 mg/kg) to assess the efficacy of laquinimod alone or in combination with DMF. MOG-induced Experimental Autoimmune Encephalomyelitis (EAE) in the C57BL/6 strain of mice is an established EAE model to test the efficacy of the candidate molecule for MS treatment.

Procedure

Disease is induced in all mice by the injection of the encephalitogenic emulsion (MOG/CFA) and intraperitoneal injection of pertussis toxin on the first day and 48 hours later.
DMF at dose levels of 25 mg/kg (sub optimal) and 50 mg/kg (optimal) are administered by the oral route, once daily (QD).
Laquinimod at dose levels of 0.12 and 0.06 mg/kg are administered by the oral route, once daily (QD).
Both DMF and laquinimod are administered prophylactic from disease induction—Day 1 until termination of the study.

Induction of EAE:

EAE is induced by subcutaneous injection of encephalitogenic emulsion at a volume of 0.2 ml/mouse in the right flank. On the day of induction, pertussis toxin is injected i.p. at a volume dose of 0.2 ml/mouse. The injection of the pertussis toxin is repeated after 48 hours.

Test Procedure:

Day 0: Subcutaneous injection of MOG into right flank, ip injection of Pertussis toxin, beginning of daily laquinimod treatment.

Day 2: ip injection of Pertussis toxin.

Day 10: initiation of scoring of mice for EAE clinical signs.

Day 30: termination of study.

Materials:
1. DMF
2. Laquinimod
3. Mycobacterium tuberculosis (MT), Difco
4. Pertussis toxin, Sigma
5. MOG 35-55, Manufactured: Novatide
6. Complete Freund's Adjuvant (CFA), Sigma
7. Saline, Manufactured: DEMO S.A
8. Sterile double distilled water (DDW)

Experimental Animals:

Healthy, nulliparous, non-pregnant female mice of the C57BL/6 strain obtained from Harlan Animal Breeding Center, Israel are used in the study.

The animals weighed 18-22 gr, and are approximately 8 weeks old on receipt.

The body weights of the animals are recorded on the day of delivery.

Overtly healthy animals are assigned to study groups arbitrarily before treatment commenced.

The mice are individually identified by using ear tags. A color-coded card on each cage gives information including cage number, group number and identification.

EAE Induction:

EAE is induced by injecting the encephalitogenic mixture (emulsion) consisting of MOG (150.0 μg/mouse) and CFA containing M. tuberculosis (2 mg MT/mL CFA).

A volume of 0.2 ml of emulsion is injected subcutaneously into the flanks of the mice.

Pertussis toxin in 0.2 ml dosage volume is injected intraperitoneally on the day of induction and 48 hours later (total amount will be 0.1+0.1=0.2 μg/mouse).

Study Design:

The mice are allocated randomly into groups according to Table 2 below.

TABLE 2

| | Treatment | | | |
|---|---|---|---|---|
| | Groups | | | |
| | (treatment | | Administration | |
| Group | initiation) | dose/day | Route | Regimen |
| 1 | Vehicle | 10 ml/kg | Oral, QD | Both |
| 2 | Laquinimod | 0.06 mg/kg | Oral, QD | DMF and |
| 2 | Laquinimod | 0.12 mg/kg | Oral, QD | Laquinimod |
| 4 | DMF | 50 mg/kg | Oral, QD | from |
| 5 | DMF | 25 mg/kg | Oral, QD | Day 1 |
| 6 | Laquinimod + | 0.06 mg/kg + | Oral (QD) + | to 30 daily |
| | DMF | 25 mg/kg | Oral (QD) | |
| 7 | Laquinimod + | 0.06 mg/kg + | Oral (QD) + | |
| | DMF | 50 mg/kg | Oral (QD) | |

Preparation and Administration of Encephalitogenic Emulsion:

Oil Portion:

20 mg MT is added to 20 ml CFA to yield 1+1=2 mg/ml MT).

Liquid Portion:

15 mg MOG or equivalent is diluted in 10 ml Normal Saline to yield 1.5 mg/ml MOG stock solution.

The emulsion is made from equal parts of oil and liquid portions (1:1) in two syringes connected to each other with Leur lock to yield 0.75 mg/ml and 1 mg/ml MT. The emulsion is transferred to insulin syringe and 0.2 ml is injected to the right flank of each mouse. Dose=0.15 mg MOG and 0.2 mg MT/mouse.

Preparation and Administration of Pertussis Toxin:

50 μL Pertussis toxin (200 μg/ml) is added to 19.95 ml saline to yield 500 ng/ml. The pertussis toxin is administered intraperitoneally on the day of encephalitogen injection and 48 hours later (100.0 ng/0.2 ml/mouse). Total 200 ng/mouse.

Preparation and Administration of Test Articles

DMF Formulations: 0.08% Methocel/H₂O

A concentration of 2.5 and 5 mg/ml for dose levels of 25 and 50 mg/kg respectively. The mice are administered with the two concentrations of DMF (2.5 and 5 mg/ml) a volume dose level of 200 μl/mouse by the oral route for dose levels of 25 and 50 mg/kg respectively.

Laquinimod Formulations:

A concentration of 0.006 and 0.012 mg/ml laquinimod is prepared in DDW. The test formulations are stored at 2 to 8° C. until use in amber colored bottles.

The mice are administered with the two concentrations of laquinimod (0.006 and 0.012 mg/ml) a volume dose level of 200 μl/mouse by the oral route for dose levels of 0.06 and 0.12 mg/kg respectively. Both the DMF and the laquinimod formulations are administered from Day 1, once daily (QD). Six hours interval is maintained daily between administration of laquinimod and DMF.

EAE Clinical Signs:

The mice are observed daily from the 10th day post-EAE induction (first injection of MOG) and the EAE clinical signs are scored according to the grades described in Table 3 presented below.

TABLE 3
Evaluation of the EAE clinical signs

| Score | Signs | Description |
|---|---|---|
| 0 | Normal | No neurological signs. |
| | behavior | |
| 1 | Limp tail | Part or the whole tail is limp and droopy. |
| 2 | righting reflex | Animal has difficulties rolling onto his feet |
| | | when laid on its back |
| 3 | Hind leg | wobbly walk - when the mouse walks the hind |
| | weakness | legs are unsteady |
| 4 | Hind leg | The mouse drags its hind legs but is able to |
| | paralysis | move around using its fore legs |
| 5 | Full paralysis | The mouse can't move around, it looks thinner |
| | | and emaciated. |
| 6 | Moribund/Death | |

All mice with score 1 and above are considered sick. When the first clinical sign appears all mice are given food soaked in water, which is spread on different places on the bedding of the cages.

Interpretation of Results

Calculation of the Incidence of Disease (Disease Ratio)
The number of sick animals in each group is summed.
The incidence of disease is calculated as

$$INCIDENCE of DISEASE = ( \frac{No . of sick mice in treated group}{No . of sick mice in treated group} )$$

The percent inhibition according to incidence is calculated as

$$INHIBITION ( \% ) of INCIDENCE = ( 1 - \frac{Number of sick mice in treated group}{Number of sick mice in treated group} ) \times 100$$

Calculation of the Mortality/Moribundity Rate (Mortality Ratio)
The number of dead or moribund animals in each group is summed.
The mortality of disease is calculated as

$$MORTALITY of DISEASE = ( \frac{No . of dead or moribound mice in treated group}{No . of dead or moribound mice in control group} )$$

The percent inhibition according to mortality is calculated as

$$INHIBITION ( \% ) of MORTALITY = ( 1 - \frac{No . of dead or moribound mice in treated group}{No . of dead or moribound mice in control group} ) \times 100$$

Calculation of Duration of Disease
The mean duration of disease expressed in days is calculated as

$$MeanDuration = ( \frac{Σ Duration of disease of each mouse}{No . of mice in the group} )$$

Calculation of Mean Delay in Onset of Disease
The mean onset of disease expressed in days is calculated as

$$MeanOnset = ( \frac{Σ Onset of disease of each mouse}{No . of mice in the group} )$$

The mean delay in onset of disease expressed in days is calculated by subtracting the mean onset of disease in control group from test group.

Calculation of the Mean Maximal Score and Percent Inhibition
The mean maximal score (MMS) of each group is calculated as

$$MMS = ( \frac{Σ Maximal sc ore of each mouse}{No . of mice in the group} )$$

The percent inhibition according to MMS is calculated as

$$INHIBITION ( \% ) of MMS = ( 1 - \frac{MMS of treated group}{MMS of control group} ) \times 100$$

Calculation of the Group Mean Score and Percent Inhibition
The daily scores of each mouse in the test group are summed and the individual mean daily score (IMS) is calculated as

$$IMS = ( \frac{Σ Daily score of mouse}{Observation period ( days )} )$$

The mean group score (GMS) is calculated as

$$GMS = ( \frac{Σ IMS of each mouse}{No . of mice in the group} )$$

The percent inhibition is calculated as

$$INHIBITION ( \% ) of GMS = ( 1 - \frac{GMS of treated group}{GMS of control group} ) \times 100$$

### RESULTS & CONCLUSIONS

In groups of mice, a total blocking of EAE in the group treated with DMF at optimal dose level of 50 mg/kg in combination with 0.06 mg/kg dose of laquinimod exhibits therapeutic activity at least as effective as the optimal dose of DMF (50 mg/kg) alone and 0.12 mg/kg dose of laquinimod alone according to GMS when compared to the vehicle administered control group.

In groups of mice, a total blocking of EAE in the group treated with DMF at optimal dose level of 50 mg/kg in combination with 0.06 mg/kg dose of laquinimod exhibits therapeutic activity superior to the optimal dose of DMF (50 mg/kg) alone and 0.12 mg/kg dose of laquinimod alone according to GMS when compared to the vehicle administered control group.

In groups of mice, a total blocking of EAE in the group treated with DMF at suboptimal dose level of 25 mg/kg in combination with 0.06 mg/kg dose of laquinimod exhibits activity at least as effective as the optimal dose of DMF (50 mg/kg) alone and 0.12 mg/kg dose of laquinimod alone according to GMS when compared to the vehicle administered control group.

In groups of mice, a total blocking of EAE in the group treated with DMF at suboptimal dose level of 25 mg/kg in combination with 0.06 mg/kg dose of laquinimod exhibits activity superior to the optimal dose of DMF (50 mg/kg) alone and 0.12 mg/kg dose of laquinimod alone according to GMS when compared to the vehicle administered control group.

In this study, each compound alone shows a dose dependent inhibition of disease severity. However, while the lower dosages tested (0.06 mg/kg laquinimod and 25 m/kg DMF) are moderately effective individually; the combination of DMF and laquinimod when each is administered at the respective lower dosage is so potent that it completely abrogated disease. This unexpected result suggests that lower dosages of laquinimod and DMF can be used in combination to achieve a greater than additive therapeutic result, and provides evidence that such a combination can be used for therapeutic treatment of human MS and CIS patients.

### Example 1B: Assessment of Efficacy of Laquinimod in Combination with DMF in MOG-Induced EAE

The objective of this study was to assess the effect of combining laquinimod and DMF treatments in MOG induced EAE. The C57BL/6 strain of mouse was selected, as it is an established chronic EAE model to test for the efficacy of candidate molecules for the treatment of MS.

Materials and Methods

Disease was induced in all mice by the injection of the encephalitogenic emulsion (MOG/CFA). The test articles and vehicle were dosed daily via gavage from Day 1 until Day 30 (termination of study).

Materials:

Materials included dimethyl fumarate (Sigma), laquinimod, Pertusis toxin (Sigma, Code #2980), Myelin Oligodendrocyte Lipoprotein (Novatide, MOG-35-55), Complete Freund's Adjuvant (CFA) (Sigma, Code F5881), Mycobacterium tuberculosis H37RA MT, (Difco, Code 231141), and Methocel (methylcellulose (MC)) (Sigma, M7140-500G).

Healthy, nulliparous, non-pregnant female mice of the C57BL/6 Strain were used. The animals weighed 17-20 g on arrival, and were approximately 11 weeks of age at the time of induction. The body weights of the animals were recorded on the day of delivery. Overtly healthy animals were assigned to study groups arbitrarily before treatment commenced.

The mice were individually identified by markings on the body. Information including cage number, group number and identification were provided in a color-coded card on each cage. The test formulations were prepared by one researcher and the treatment and scoring procedure is carried out by a different researcher blind to the identification of the treatment groups.

EAE Induction:

Active EAE was induced on Day 1 via subcutaneous injection in the flanks at two injection sites. The encephalitogenic mixture (emulsion) consisting of MOG and commercial CFA containing 2 mg/mL Mycobacterium tuberculosis (MT) at a volume of 0.2 mL/mouse was injected in the right flank of the animals. Pertussis toxin was injected intraperitoneally on the day of induction and 48 hours later at dose level of 100 ng/0.2 ml/mouse. The dose of the MOG and MT was 150 μg/mouse and 200 μg/mouse respectively.

Preparation and Administration of Encephalitogenic Emulsion:

Oil Portion:

CFA (containing 1 mg/ml MT) enriched with mycobacterium tuberculosis to yield 2 mg/ml MT.

Liquid Portion:

38 mg MOG or equivalent was dissolved in 25.33 ml

Normal saline to yield 1.5 mg/ml MOG.

Emulsion:

The emulsions was made from equal parts of oil (CFA containing 2.0 mg/ml MT) and liquid portions (1.5 mg MOG) in two syringes connected to each other with Leur lock to yield 0.75 mg/ml MOG. The emulsion was administered to mice of the respective groups once on Day 1 via subcutaneously injection at two injection sites (in the flanks of the mice).

The dose of the MOG in all the groups was 0.15 mg/0.2 ml/mouse. The dose of the MT in all the groups was 0.2 mg/0.2 ml/mouse.

Preparation and Administration of Pertussis Toxin:

55.0 μl Pertussis toxin (200 μg/ml) or equivalent was added to 21.945 ml saline to yield 0.5 μg/ml. 0.2 ml of 0.5 μg/ml Pertussis toxin solution was injected intraperitoneally immediately after the MOG emulsion injection for a dose level of 100 ng/mouse. Injection of the pertussis toxin was repeated in a similar manner after 48 hours.

Group Assignment:

On Day 1 the MOG EAE induced mice were allocated to the following treatment groups (15 mice/group):

TABLE 4
Group Assignment for Experiment 1B

| | Treatment | | Administration | Admin. |
|---|---|---|---|---|
| Group | groups | Dose/day | Route | Period |
| 1 | Vehicle (0.08% | 0.2 ml/mouse | Gavage bid(AM/PM) | From Day |
| | MC) | | | 1 to 30 |
| 2 | Laquinimod | 5 mg/kg/day | Gavage qd (AM) | From Day |
| | 0.08% MC | 0.2 ml/mouse | Gavage qd (PM) | 1 to 30 |
| 3 | Laquinimod | 10 mg/kg/day | Gavage qd (AM) | From Day |
| | 0.08% MC | 0.2 ml/mouse | Gavage qd (PM) | 1 to 30 |
| 4 | Laquinimod | 25 mg/kg/day | Gavage qd (AM) | From Day |
| | 0.08% MC | 0.2 ml/mouse | Gavage qd (PM) | 1 to 30 |
| 5 | DMF | 45 mg/kg => 90 mg/kg/day | Gavage bid (AM/PM) | From Day |
| | | | | 1 to 30 |
| 6* | DMF | 45 mg/kg => 90 mg/kg/day | Gavage bid(AM/PM) | From Day |
| | Laquinimod | 5 mg/kg/day | Gavage qd (AM) | 1 to 30 |
| 7* | DMF | 45 mg/kg =>90 mg/kg/day | Gavage bid(AM/PM) | From Day |
| | Laquinimod | 10 mg/kg/day | Gavage qd (AM) | 1 to 30 |

*DMF was suspended in laquinimod solution in the morning treatment
**AM/PM indicates morning/afternoon.

Test Formulations:

Laquinimod:

Laquinimod was diluted in 0.08% Methocel/H2O. For dose level of 25.0 mg/kg laquinimod, 2.5 mg/ml stock solution was prepared (group 4). For dose level of 10.0 mg/kg laquinimod, 1.0 mg/ml stock solution was prepared (groups 3 and 7). For dose level of 5.0 mg/kg laquinimod, 0.5 mg/ml stock solution was prepared (groups 2 and 6). Laquinimod was administered to the respective groups daily, by oral gavage at a volume of 0.2 ml/mouse. Laquinimod was administered from the initiation of the study, daily to mice of groups 2, 3, 4, 6 and 7. The test formulations were stored at 2 to 8° C. until use in amber colored bottles.

DMF:

Formulation for group 5 was diluted in 0.08% Methocel/H₂O to yield a concentration of 4.5 mg/ml for dose level of 45 mg/kg. The mice were administered with DMF at volume dose level of 200 μl/mouse by the oral gavage route twice a day for a total dose level of 90 mg/kg/day.

DMF and Laquinimod Combined:

For the morning (AM) gavage (groups and 7), 4.5 mg of DMF were suspended for every 1 ml of laquinimod solution. (From the stock solutions made of laquinimod 1.0 or 0.5 mg/ml diluted in 0.08% Methocel/H2O solutions.)

Treatments:

Mice of all the treatment groups were administered the respective test formulation from Day 1, twice daily (bid) according to experimental design.

Experimental Observations

Morbidity and Mortality:

All animals were examined once daily to detect if any are moribund. Mice were weighed once weekly.

EAE Clinical Signs:

The mice were observed daily from the 8^{th} day post EAE-induction and EAE clinical signs were scored. The scores were recorded on observation cards according to the grades described in Table 3 shown above.

All mice with score 1 and above were considered sick. When the first clinical sign appears all mice were given food soaked in water, which was spread on different places on the bedding of the cages. For calculation purposes, the score of animals that were sacrificed or died was carried forward.

Interpretation of Results:

Same as in Experiment 1A.

Results:

A summary of the incidence, mortality, MMS, GMS, duration of the disease, onset of the disease and the activity of each group compared to the vehicle treated control group are shown in the Summarized Table 5 below:

TABLE 5

| | | | | | | | | Mean | Mean |
|---|---|---|---|---|---|---|---|---|---|
| | | | % | MMS | % | GMS | % | Onset | Duration |
| Group | Mortality | Incidence | Inhibition 1 | value | Inhibition 2 | value | Inhibition 3 | (days) | (days) |
| 1 | 0/15 | 15/15 | — | 3.5 ± 1.0 | — | 2.1 ± 0.8 | — | 13.5 ± 1.6 | 17.0 ± 2.2 |
| 2 | 0/15 | 10/15 | 33.3 | 2.1 ± 1.7 | 40.0 | 0.8 ± 0.7 | 61.9 | 22.7 ± 6.4 | 8.0 ± 6.2 |
| | | | | | P = 0.019 | | P < 0.001 | P < 0.001 | P < 0.001 |
| 3 | 0/15 | 4/15 | 73.3 | 0.6 ± 0.2 | 82.9 | 0.2 ± 0.5 | 90.5 | 28.9 ± 3.8 | 1.8 ± 3.7 |
| | | | | | P < 0.001 | | P < 0.001 | P < 0.001 | P < 0.001 |
| 4 | 0/15 | 0/15 | 100.0 | 0.0 ± 0.0 | 100.0 | 0.0 ± 0.0 | 100.0 | 0.0 ± 0.0 | 0.0 ± 0.0 |
| | | | | | P < 0.001 | | P < 0.001 | P < 0.001 | P < 0.001 |
| 5 | 0/15 | 13/15 | 13.3 | 2.6 ± 1.4 | 25.7 | 1.4 ± 0.9 | 33.3 | 17.1 ± 6.3 | 13.4 ± 6.2 |
| | | | | | P = 0.074 | | P = 0.061 | P = 0.037 | P = 0.067 |
| 6 | 0/15 | 4/15 | 73.3 | 0.4 ± 0.7 | 88.6 | 0.1 ± 0.2 | 95.2 | 29.0 ± 4.1 | 2.0 ± 4.1 |
| | | | | | P < 0.001 | | P < 0.001 | P < 0.001 | P < 0.001 |
| 7 | 0/15 | 1/15 | 93.3 | 0.3 ± 1.0 | 93.4 | 0.1 ± 0.5 | 95.2 | 30.1 ± 3.4 | 0.9 ± 3.4 |
| | | | | | P < 0.001 | | P < 0.001 | P < 0.001 | P < 0.001 |

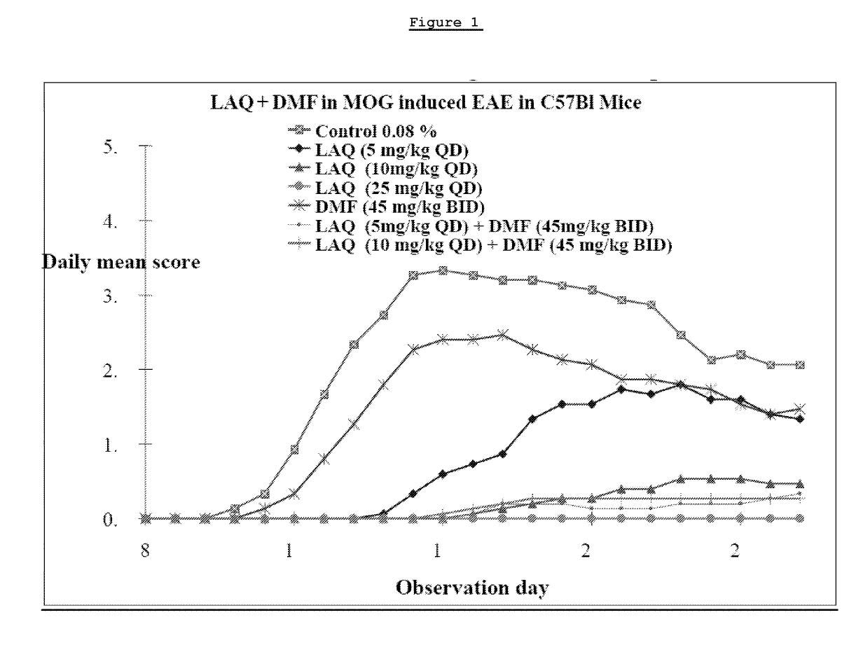

The clinical profile of the treatment groups are presented graphically in FIG. 1.

Under the conditions of the test, DMF at dose level of 45 mg/kg mouse (BID) exhibited additive activity in the suppression of EAE when tested in combination with laquinimod at dose level of 5 mg/kg. The group treated with DMF at dose level of 45 mg/kg (BID) in combination with laquinimod (5 mg/kg) exhibited 95.2% (p<0.001) activity according to GMS compared to 33.3% activity (p=0.061) in the group treated with DMF at dose level of 45 mg/kg (BID) and 61.9% activity (p<0.001) in the group treated with laquinimod at dose level of 5 mg/kg when compared to the vehicle administered control group.

The group treated with DMF at dose level of 45 mg/kg (BID) in combination with laquinimod (10 mg/kg) exhibited 95.2% activity (p<0.001) according to GMS compared to 33.3% activity (p=0.061) in the group treated with DMF at dose level of 45 mg/kg (BID) and 90.5% (p<0.001) activity in the group treated with laquinimod at dose level of 10 mg/kg when compared to the vehicle administered control group.

Laquinimod at dose level of 25 mg/kg (QD) exhibited 100% activity (p<0.001) according to GMS when compared to the vehicle administered control group.

### Example 2A: Assessment of Daily Administration of Laquinimod (0.3 mg) as an Add-on Therapy to a Human Patient Already Receiving DMF

Daily administration of laquinimod (p.o., 0.3 mg/day) as an add-on therapy for a human patient already receiving DMF (120, 240, 360, 480, or 720 mg/day) provides improved efficacy (provides at least the same effect with fewer adverse side effects, or an additive or more than an additive effect without unduly increasing adverse side effects or affecting the safety of the treatment) in relapsing multiple sclerosis (RMS) subjects compared to administration of the same level of DMF alone.

### Example 2B: Assessment of Daily Administration of Laquinimod (0.6 mg) as an Add-on Therapy to a Human Patient Already Receiving DMF

Daily administration of laquinimod (p.o., 0.6 mg/day) as an add-on therapy for a human patient already receiving DMF (120, 240, 360, 480, or 720 mg/day) provides improved efficacy (provides at least the same effect with fewer adverse side effects, or an additive or more than an additive effect without unduly increasing adverse side effects or affecting the safety of the treatment) in relapsing multiple sclerosis (RMS) subjects compared to administration of the same level of DMF alone.

### Example 2C: Assessment of Daily Administration of DMF as an Add-on Therapy to a Human Patient Already Receiving Laquinimod (0.3 mg)

Daily administration of DMF (120, 240, 360, 480, or 720 mg/day) as an add-on therapy for a human patient already receiving a suboptimal dosage of laquinimod (0.3 mg) provides improved efficacy (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg) of laquinimod alone.

### Example 3A: Assessment of Efficacy of Laquinimod as an Add-on Therapy to a Human Patient Already Receiving DMF to Reduce Brain Atrophy

Daily administration of laquinimod (p.o., 0.3 mg/day or 0.6 mg/day) as an add-on therapy for a human patient already receiving DMF (120, 240, 360, 480, or 720 mg/day) provides improved efficacy in reducing brain atrophy (provides at least the same effect with fewer adverse side effects, or an additive or more than an additive effect without unduly increasing adverse side effects or affecting the safety of the treatment) in relapsing multiple sclerosis (RMS) subjects compared to administration of the same level of DMF alone.

### Example 3B: Assessment of Efficacy of DMF as an Add-on Therapy to a Human Patient Already Receiving Laquinimod to Reduce Brain Atrophy

Daily administration of DMF (120, 240, 360, 480, or 720 mg/day) as an add-on therapy for a human patient already receiving a suboptimal dosage of laquinimod (0.3 mg) reduces the amount of brain atrophy over 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg) of laquinimod alone.

### Example 4A: Assessment of Efficacy of Laquinimod as an Add-on Therapy to a Human Patient Already Receiving DMF to Reduce the Rate of Development of Clinically Definite MS and Preventing Irreversible Brain Damage

Daily administration of laquinimod (p.o., 0.3 mg/day or 0.6 mg/day) as an add-on therapy for a human patient already receiving DMF (120, 240, 360, 480, or 720 mg/day) provides a clinically meaningful advantage and is more effective (provides an additive or more than an additive effect without unduly increasing adverse side effects or affecting the safety of the treatment) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons compared to administration of the same level of DMF alone.

### Example 4B: Assessment of Efficacy of DMF as an Add-on Therapy to a Human Patient Already Receiving Laquinimod to Reduce the Rate of Development of Clinically Definite MS and Preventing Irreversible Brain Damage

Daily administration of DMF (120, 240, 360, 480, or 720 mg/day) as an add-on therapy for a human patient already receiving a suboptimal dosage of laquinimod (0.3 mg) provides a clinically meaningful advantage and is more effective (provides an additive or more than an additive effect without unduly increasing adverse side effects or affecting the safety of the treatment) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons compared to administration of an higher dosage (0.6 mg) of laquinimod alone.

### Example 5A: Assessment of Efficacy of Laquinimod as an Add-on Therapy to a Human Patient Already Receiving DMF to Reduce Cumulative Number of New T1 Gd-Enhancing Lesions

Daily administration of laquinimod (p.o., 0.3 mg/day or 0.6 mg/day) as an add-on therapy for a human patient already receiving DMF (120, 240, 360, 480, or 720 mg/day) reduces the cumulative number of new T1 Gd-enhancing lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the same level of DMF alone.

### Example 5B: Assessment of Efficacy of DMF as an Add-on Therapy to a Human Patient Already Receiving Laquinimod to Reduce Cumulative Number of New T1 Gd-Enhancing Lesions

Daily administration of DMF (120, 240, 360, 480, or 720 mg/day) as an add-on therapy for a human patient already receiving a suboptimal dosage of laquinimod (0.3 mg) reduces the cumulative number of new T1 Gd-enhancing lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg) of laquinimod alone.

### Example 6A: Assessment of Efficacy of Laquinimod as an Add-on Therapy to a Human Patient Already Receiving DMF to Reduce Cumulative Number of New T2 Lesions

Daily administration of laquinimod (p.o., 0.3 mg/day or 0.6 mg/day) as an add-on therapy for a human patient already receiving DMF (120, 240, 360, 480, or 720 mg/day) reduces the cumulative number of new T2 lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the same level of DMF alone.

### Example 6B: Assessment of Efficacy of DMF as an Add-on Therapy to a Human Patient Already Receiving Laquinimod to Reduce Cumulative Number of New T2 Lesions

Daily administration of DMF (120, 240, 360, 480, or 720 mg/day) as an add-on therapy for a human patient already receiving a suboptimal dosage of laquinimod (0.3 mg) reduces the cumulative number of new T2 lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg) of laquinimod alone.

### Example 7A: Assessment of Efficacy of Laquinimod as an Add-on Therapy to a Human Patient Already Receiving DMF to Reduce Cumulative Number of New T1 Hypointense Lesions

Daily administration of laquinimod (p.o., 0.3 mg/day or 0.6 mg/day) as an add-on therapy for a human patient already receiving DMF (120, 240, 360, 480, or 720 mg/day) reduces the cumulative number of new T1 hypointense lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the same level of DMF alone.

### Example 7B: Assessment of Efficacy of DMF as an Add-on Therapy to a Human Patient Already Receiving Laquinimod to Reduce Cumulative Number of New T1 Hypointense Lesions

Daily administration of DMF (120, 240, 360, 480, or 720 mg/day) as an add-on therapy for a human patient already receiving a suboptimal dosage of laquinimod (0.3 mg) reduces the cumulative number of new T1 hypointense lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg) of laquinimod alone.

### Example 8A: Assessment of Efficacy of Laquinimod as an Add-on Therapy to a Human Patient Already Receiving DMF to Reduce Total Volume of T1 Gd-Enhancing Lesions

Daily administration of laquinimod (p.o., 0.3 mg/day or 0.6 mg/day) as an add-on therapy for a human patient already receiving DMF (120, 240, 360, 480, or 720 mg/day) reduces the total volume of T1 Gd-enhancing lesions as measured at 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the same level of DMF alone.

### Example 8B: Assessment of Efficacy of DMF as an Add-on Therapy to a Human Patient Already Receiving Laquinimod to Reduce Total Volume of T1 Gd-Enhancing Lesions

Daily administration of DMF (120, 240, 360, 480, or 720 mg/day) as an add-on therapy for a human patient already receiving a suboptimal dosage of laquinimod (0.3 mg) reduces the total volume of T1 Gd-enhancing lesions as measured at 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg) of laquinimod alone.

### Example 9A: Assessment of Efficacy of Laquinimod as an Add-on Therapy to a Human Patient Already Receiving DMF to Reduce Total Volume of T2 Lesions

Daily administration of laquinimod (p.o., 0.3 mg/day or 0.6 mg/day) as an add-on therapy for a human patient already receiving DMF (120, 240, 360, 480, or 720 mg/day) reduces the total volume of T2 lesions as measured at 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the same level of DMF alone.

### Example 9B: Assessment of Efficacy of DMF as an Add-on Therapy to a Human Patient Already Receiving Laquinimod to Reduce Total Volume of T2 Lesions

Daily administration of DMF (120, 240, 360, 480, or 720 mg/day) as an add-on therapy for a human patient already receiving a suboptimal dosage of laquinimod (0.3 mg) reduces the total volume of T2 lesions as measured at 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg) of laquinimod alone.

### Example 10A: Assessment of Efficacy of Laquinimod as an Add-on Therapy to a Human Patient Already Receiving DMF to Reduce Annualized Relapse Rate

Daily administration of laquinimod (p.o., 0.3 mg/day or 0.6 mg/day) as an add-on therapy for a human patient already receiving DMF (120, 240, 360, 480, or 720 mg/day) reduces annualized relapse rate (provides at least the same effect with fewer adverse side effects, or an additive or more than an additive effect without unduly increasing adverse side effects or affecting the safety of the treatment) in relapsing multiple sclerosis (RMS) subjects compared to administration of the same level of DMF alone.

### Example 10B: Assessment of Efficacy of DMF as an Add-on Therapy to a Human Patient Already Receiving Laquinimod to Reduce Annualized Relapse Rate

Daily administration of DMF (120, 240, 360, 480, or 720 mg/day) as an add-on therapy for a human patient already receiving a suboptimal dosage of laquinimod (0.3 mg) reduces annualized relapse rate (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg) of laquinimod alone.

### Example 11A: Assessment of Efficacy of Laquinimod as an Add-on Therapy to a Human Patient Already Receiving DMF to Reduce Accumulation of Physical Disability

Daily administration of laquinimod (p.o., 0.3 mg/day or 0.6 mg/day) as an add-on therapy for a human patient already receiving DMF (120, 240, 360, 480, or 720 mg/day) reduces accumulation of physical disability (provides at least the same effect with fewer adverse side effects, or an additive or more than an additive effect without unduly increasing adverse side effects or affecting the safety of the treatment) in relapsing multiple sclerosis (RMS) subjects compared to administration of the same level of DMF alone.

### Example 11B: Assessment of Efficacy of DMF as an Add-on Therapy to a Human Patient Already Receiving Laquinimod to Reduce Accumulation of Physical Disability

Daily administration of DMF (120, 240, 360, 480, or 720 mg/day) as an add-on therapy for a human patient already receiving a suboptimal dosage of laquinimod (0.3 mg) reduces accumulation of physical disability (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg) of laquinimod alone.

### Example 12A: Assessment of Efficacy of Laquinimod as an Add-on Therapy to a Human Patient Already Receiving DMF to Delay the Conversion to Clinically Definite MS

Daily administration of laquinimod (p.o., 0.3 mg/day or 0.6 mg/day or 1.2 mg/day) as an add-on therapy for a human patient already receiving DMF (120, 240, 360, 480, or 720 mg/day) provides a clinically meaningful advantage and is more effective (provides at least the same effect with fewer adverse side effects, or an additive or more than an additive effect without unduly increasing adverse side effects or affecting the safety of the treatment) in delaying the conversion to clinically definite MS in patients presenting a CIS suggestive of MS compared to administration of the same level of DMF alone.

### Example 12B: Assessment of Efficacy of DMF as an Add-on Therapy to a Human Patient Already Receiving Laquinimod to Delay the Conversion to Clinically Definite MS

Daily administration of DMF (120, 240, 360, 480, or 720 mg/day) as an add-on therapy for a human patient already receiving a suboptimal dosage of laquinimod (0.3 mg) provides a clinically meaningful advantage and is more effective (provides at least the same effect with fewer adverse side effects, or an additive or more than an additive effect without unduly increasing adverse side effects or affecting the safety of the treatment) in delaying the conversion to clinically definite MS in patients presenting a CIS suggestive of MS compared to administration of a higher dosage (0.6 mg) of laquinimod alone.

### Example 13A: Assessment of Efficacy of Laquinimod as an Add-on Therapy to a Human Patient Already Receiving DMF to Reduce the Number of Adverse Events

Daily administration of laquinimod (p.o., 0.3 mg/day or 0.6 mg/day or 1.2 mg/day) as an add-on therapy for a human patient already receiving DMF (120, 240, 360, 480, or 720 mg/day) reduces the number of adverse events over a period of 2, 4 or 6 months (provides at least the same effect with fewer adverse side effects, or an additive or more than an additive effect with fewer adverse side effects) compared to administration of the same level of DMF alone.

### Example 13B: Assessment of Efficacy of DMF as an Add-on Therapy to a Human Patient Already Receiving Laquinimod to Reduce the Number of Adverse Events

Daily administration of DMF (120, 240, 360, 480, or 720 mg/day) as an add-on therapy for a human patient already receiving a suboptimal dosage of laquinimod (0.3 mg) reduces the number of adverse events over a period of 2, 4 or 6 months (provides at least the same effect with fewer adverse side effects, or an additive or more than an additive effect with fewer adverse side effects) in relapsing multiple sclerosis (RMS) subjects compared to administration of a higher dosage (0.6 mg) of laquinimod alone.

### Example 14: Assessment of Efficacy of Daily Administration of Laquinimod and DMF as a Combination Therapy for a Human Patient to Reduce Brain Atrophy

Daily administration of laquinimod (p.o., 0.3 mg/day or 0.6 mg/day or 1.2 mg/day) and DMF (120, 240, 360, 480, or 720 mg/day) as a combination therapy for a human patient reduces the amount of brain atrophy over 6 months and provides at least the same effect with fewer adverse side effects, or an additive or more than an additive effect without unduly increasing adverse side effects or affecting the safety of the treatment compared to administration of the same level of DMF alone.

### Example 15: Assessment of Efficacy of Daily Administration of Laquinimod and DMF as a Combination Therapy for a Human Patient to Reduce Cumulative Number of New T1 Gd-Enhancing Lesions

Daily administration of laquinimod (p.o., 0.3 mg/day or 0.6 mg/day or 1.2 mg/day) and DMF (120, 240, 360, 480, or 720 mg/day) as a combination therapy for a human patient reduces the cumulative number of new T1 Gd-enhancing lesions as measured at 2, 4 and 6 months and provides at least the same effect with fewer adverse side effects, or an additive or more than an additive effect without unduly increasing adverse side effects or affecting the safety of the treatment compared to administration of the same level of DMF alone.

### Example 16: Assessment of Efficacy of Daily Administration of Laquinimod and DMF as a Combination Therapy for a Human Patient to Reduce Cumulative Number of New T2 Lesions

Daily administration of laquinimod (p.o., 0.3 mg/day or 0.6 mg/day or 1.2 mg/day) and DMF (120, 240, 360, 480, or 720 mg/day) as a combination therapy for a human patient reduces the cumulative number of new T2 lesions as measured at 2, 4 and 6 months and provides at least the same effect with fewer adverse side effects, or an additive or more than an additive effect without unduly increasing adverse side effects or affecting the safety of the treatment compared to administration of the same level of DMF alone.

### Example 17: Assessment of Efficacy of Daily Administration of Laquinimod and DMF as a Combination Therapy for a Human Patient to Reduce Cumulative Number of New T1 Hypointense Lesions

Daily administration of laquinimod (p.o., 0.3 mg/day or 0.6 mg/day or 1.2 mg/day) and DMF (120, 240, 360, 480, or 720 mg/day) as a combination therapy for a human patient reduces the cumulative number of new T1 hypointense lesions as measured at 2, 4 and 6 months and provides at least the same effect with fewer adverse side effects, or an additive or more than an additive effect without unduly increasing adverse side effects or affecting the safety of the treatment compared to administration of the same level of DMF alone.

### Example 18: Assessment of Efficacy of Daily Administration of Laquinimod and DMF as a Combination Therapy for a Human Patient to Reduce Total Volume of T1 Gd-Enhancing Lesions

Daily administration of laquinimod (p.o., 0.3 mg/day or 0.6 mg/day or 1.2 mg/day) and DMF (120, 240, 360, 480, or 720 mg/day) as a combination therapy for a human patient reduces the total volume of T1 Gd-enhancing lesions as measured at 6 months and provides at least the same effect with fewer adverse side effects, or an additive or more than an additive effect without unduly increasing adverse side effects or affecting the safety of the treatment compared to administration of the same level of DMF alone.

### Example 19: Assessment of Efficacy of Daily Administration of Laquinimod and DMF as a Combination Therapy for a Human Patient to Reduce Total Volume of T2 Lesions

Daily administration of laquinimod (p.o., 0.3 mg/day or 0.6 mg/day or 1.2 mg/day) and DMF (120, 240, 360, 480, or 720 mg/day) as a combination therapy for a human patient reduces the total volume of T2 lesions as measured at 6 months and provides at least the same effect with fewer adverse side effects, or an additive or more than an additive effect without unduly increasing adverse side effects or affecting the safety of the treatment compared to administration of the same level of DMF alone.

### Example 20: Assessment of Efficacy of Daily Administration of Laquinimod and DMF as a Combination Therapy for a Human Patient to Reduce Annualized Relapse Rate

Daily administration of laquinimod (p.o., 0.3 mg/day or 0.6 mg/day or 1.2 mg/day) and DMF (120, 240, 360, 480, or 720 mg/day) as a combination therapy for a human patient reduces annualized relapse rate and provides at least the same effect with fewer adverse side effects, or an additive or more than an additive effect without unduly increasing adverse side effects or affecting the safety of the treatment compared to administration of the same level of DMF alone.

### Example 21: Assessment of Efficacy of Daily Administration of Laquinimod and DMF as a Combination Therapy for a Human Patient to Reduce Accumulation of Physical Disability

Daily administration of laquinimod (p.o., 0.3 mg/day or 0.6 mg/day or 1.2 mg/day) and DMF (120, 240, 360, 480, or 720 mg/day) as a combination therapy for a human patient reduces accumulation of physical disability and provides at least the same effect with fewer adverse side effects, or an additive or more than an additive effect without unduly increasing adverse side effects or affecting the safety of the treatment compared to administration of the same level of DMF alone. Accumulation of physical disability measured by the time to confirmed progression of EDSS during the study period (A confirmed progression of EDSS is defined as a 1 point increase from baseline on EDSS score if baseline EDSS was between 0 and 5.0, or a 0.5 point increase if baseline EDSS was 5.5. Progression cannot be confirmed during a relapse.

### Example 22: Assessment of Efficacy of Daily Administration of Laquinimod and DMF as a Combination Therapy for a Human Patient to Delay the Conversion to Clinically Definite MS

Daily administration of laquinimod (p.o., 0.3 mg/day or 0.6 mg/day or 1.2 mg/day) and DMF (120, 240, 360, 480, or 720 mg/day) as a combination therapy for a human patient provides a clinically meaningful advantage and is more effective (provides at least the same effect with fewer adverse side effects, or an additive or more than an additive effect without unduly increasing adverse side effects or affecting the safety of the treatment) in delaying the conversion to clinically definite MS in patients presenting a CIS suggestive of MS than when DMF is administered alone (at the same dose).

### Example 23: Assessment of Efficacy of Daily Administration of Laquinimod and DMF as a Combination Therapy for a Human Patient to Reduce the Rate of Development of Clinically Definite MS and Preventing Irreversible Brain Damage

Daily administration of laquinimod (p.o., 0.3 mg/day or 0.6 mg/day or 1.2 mg/day) and DMF (120, 240, 360, 480, or 720 mg/day) as a combination therapy for a human patient provides a clinically meaningful advantage and is more effective (provides at least the same effect with fewer adverse side effects, or an additive or more than an additive effect without unduly increasing adverse side effects or affecting the safety of the treatment) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when DMF is administered alone (at the same dose).

### Example 24: Assessment of Adverse Events from Daily Administration of Laquinimod and DMF as a Combination Therapy for a Human Patient

Daily administration of laquinimod (p.o., 0.3 mg/day or 0.6 mg/day or 1.2 mg/day) and DMF (120, 240, 360, 480, or 720 mg/day) as a combination therapy for a human patient results in a reduced number of adverse events over a period of 2, 4 or 6 months compared to the same dose of DMF.

### Example 25: Assessment of Daily Administration of Laquinimod (0.3 Mg/Day) and DMF as a Combination Therapy for Relapsing Multiple Sclerosis (RMS) Patients

Daily administration of laquinimod (p.o., 0.3 mg/day) and DMF (120, 240, 360, 480, or 720 mg/day) provides a clinically meaningful advantage and is more effective (provides an additive effect or more than an additive effect) in treating relapsing multiple sclerosis (RMS) patients than when each agent is administered alone (at the same dose) in the following manner:
1. Daily administration of laquinimod (p.o., 0.3 mg/day) and DMF is more effective (provides an additive effect or more than an additive effect) in reducing the number of confirmed relapses and therefore the relapse rate, in relapsing multiple sclerosis (RMS) patients compared to administration of the same level of DMF alone or laquinimod (p.o., 0.6 mg/day).
2. Daily administration of laquinimod (p.o., 0.3 mg/day) and DMF is also more effective (provides an additive effect or more than an additive effect) in reducing the accumulation of physical disability in relapsing multiple sclerosis (RMS) patients, as measured by the time to confirmed progression of EDSS, compared to administration of the same level of DMF alone or laquinimod (p.o., 0.6 mg/day).
3. Daily administration of laquinimod (p.o., 0.3 mg/day) and DMF is also more effective (provides an additive effect or more than an additive effect) in reducing MRI-monitored disease activity in relapsing multiple sclerosis (RMS) patients, as measured by the cumulative number of T1 Gd-enhancing lesions on T1-weighted images, the cumulative number of new T2 lesions, change in brain volume, the cumulative number of new T1hypointense lesions on T1-weight images (black holes), presence or absence of GdE lesions, change in total volume of T1 Gd-enhancing lesions, and/or change in total volume of T2 lesions, compared to administration of the same level of DMF alone or laquinimod (p.o., 0.6 mg/day).
4. Daily administration of laquinimod (p.o., 0.3 mg/day) and DMF is more effective (provides an additive effect or more than an additive effect) in reducing brain atrophy in relapsing multiple sclerosis (RMS) patients, compared to administration of the same level of DMF alone or laquinimod (p.o., 0.6 mg/day).
5. Daily administration of laquinimod (p.o., 0.3 mg/day) and DMF is more effective (provides an additive effect or more than an additive effect) in reducing the frequency of relapses, the frequency of clinical exacerbation, the risk for confirmed progression, and the time to confirmed disease progression in relapsing multiple sclerosis (RMS) patients, compared to administration of the same level of DMF alone or laquinimod (p.o., 0.6 mg/day).

### Example 26: Assessment of Daily Administration of Laquinimod (0.6 Mg/Day) and DMF as a Combination Therapy for Relapsing Multiple Sclerosis (RMS) Patients

Daily administration of laquinimod (p.o., 0.6 mg/day) and DMF (120, 240, 360, 480, or 720 mg/day) provides a clinically meaningful advantage and is more effective (provides an additive effect or more than an additive effect) in treating relapsing multiple sclerosis (RMS) patients than when each agent is administered alone (at the same dose) in the following manner:
1. Daily administration of laquinimod (p.o., 0.6 mg/day) and DMF is more effective (provides an additive effect or more than an additive effect) in reducing the number of confirmed relapses and therefore the relapse rate, in relapsing multiple sclerosis (RMS) patients compared to administration of the same level of each agent alone.
2. Daily administration of laquinimod (p.o., 0.6 mg/day) and DMF is also more effective (provides an additive effect or more than an additive effect) in reducing the accumulation of physical disability in relapsing multiple sclerosis (RMS) patients, as measured by the time to confirmed progression of EDSS, compared to administration of the same level of each agent alone.
3. Daily administration of laquinimod (p.o., 0.6 mg/day) and DMF is also more effective (provides an additive effect or more than an additive effect) in reducing MRI-monitored disease activity in relapsing multiple sclerosis (RMS) patients, as measured by the cumulative number of T1 Gd-enhancing lesions on T1-weighted images, the cumulative number of new T2 lesions, change in brain volume, the cumulative number of new T1hypointense lesions on T1-weight images (black holes), presence or absence of GdE lesions, change in total volume of T1 Gd-enhancing lesions, and/or change in total volume of T2 lesions, compared to administration of the same level of each agent alone.
4. Daily administration of laquinimod (p.o., 0.6 mg/day) and DMF is more effective (provides an additive effect or more than an additive effect) in reducing brain atrophy in relapsing multiple sclerosis (RMS) patients, compared to administration of the same level of each agent alone.
5. Daily administration of laquinimod (p.o., 0.6 mg/day) and DMF is more effective (provides an additive effect or more than an additive effect) in reducing the frequency of relapses, the frequency of clinical exacerbation, the risk for confirmed progression, and the time to confirmed disease progression in relapsing multiple sclerosis (RMS) patients, compared to administration of the same level of each agent alone.

### Example 27: Assessment of Daily Administration of Laquinimod (1.2 mg/Day) and DMF as a Combination Therapy for Relapsing Multiple Sclerosis (RMS) Patients

Daily administration of laquinimod (p.o., 1.2 mg/day) and DMF (120, 240, 360, 480, or 720 mg/day) provides a clinically meaningful advantage and is more effective (provides an additive effect or more than an additive effect) in treating relapsing multiple sclerosis (RMS) patients than when each agent is administered alone (at the same dose) in the following manner:
6. Daily administration of laquinimod (p.o., 1.2 mg/day) and DMF is more effective (provides an additive effect or more than an additive effect) in reducing the number of confirmed relapses and therefore the relapse rate, in relapsing multiple sclerosis (RMS) patients compared to administration of the same level of each agent alone.
7. Daily administration of laquinimod (p.o., 1.2 mg/day) and DMF is also more effective (provides an additive effect or more than an additive effect) in reducing the accumulation of physical disability in relapsing multiple sclerosis (RMS) patients, as measured by the time to confirmed progression of EDSS, compared to administration of the same level of each agent alone.
8. Daily administration of laquinimod (p.o., 1.2 mg/day) and DMF is also more effective (provides an additive effect or more than an additive effect) in reducing MRI-monitored disease activity in relapsing multiple sclerosis (RMS) patients, as measured by the cumulative number of T1 Gd-enhancing lesions on T1-weighted images, the cumulative number of new T2 lesions, change in brain volume, the cumulative number of new T1hypointense lesions on T1-weight images (black holes), presence or absence of GdE lesions, change in total volume of T1 Gd-enhancing lesions, and/or change in total volume of T2 lesions, compared to administration of the same level of each agent alone.
9. Daily administration of laquinimod (p.o., 1.2 mg/day) and DMF is more effective (provides an additive effect or more than an additive effect) in reducing brain atrophy in relapsing multiple sclerosis (RMS) patients, compared to administration of the same level of each agent alone.
10. Daily administration of laquinimod (p.o., 1.2 mg/day) and DMF is more effective (provides an additive effect or more than an additive effect) in reducing the frequency of relapses, the frequency of clinical exacerbation, the risk for confirmed progression, and the time to confirmed disease progression in relapsing multiple sclerosis (RMS) patients, compared to administration of the same level of each agent alone.

### REFERENCES

1. Alejandro Horga; Xavier Montalban Jun. 4, 2008; Expert Rev Neurother. 2008; 8(5):699-714.
2. Berdyshev E V, Gorshkova I, Skobeleva A, Bittman R, Lu X, Dudek S M, Mirzapoiazova T, Garcia J G, Natarajan V. (2009). “FTY720 inhibits ceramide synthases and up-regulates dihydrosphingosine 1-phosphate formation in human lung endothelial cells.”. Journal of Biological Chemistry 284 (9): 5467-77.
3. Bjartmar C, Fox R I. (2002) “Pathological mechanisms and disease progression of multiple sclerosis: therapeutic implication”, Drugs of Today. 38:7-29.
4. Brex P A et al., (2002) “A longitudinal study of abnormalities on MRI and disability from multiple sclerosis”, N Engl J Med. Jan. 17, 2002 346(3):158-64.
5. Billich A, Bornancin F, Dévay P, Mechtcheriakova D, Urtz N, Baumruker T (2003). “Phosphorylation of the immunomodulatory drug FTY720 by sphingosine kinases”. J Biol Chem 278 (48): 47408-15.
6. Brod et al. (2000) Annals of Neurology, 47:127-131.
7. De Stefano et al. (1999) “Evidence of early axonal damage in patients with multiple sclerosis”, Neurology. 52(Suppl 2):A378.
8. Dunitz, M. Multiple sclerosis therapeutics, Ed. Rudick and Goodkin. London: Taylor & Francis, 1999.
9. EMEA Guideline on Clinical Investigation of Medicinal Products for the Treatment of Multiple Sclerosis (CPMP/EWP/561/98 Rev. 1, November. 2006).
10. Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. For Biologics Eval. and Res., Clin./Pharm., November 1999 <http://www.fda.gov/cber/gdlns/metabol.pdf>.
11. Gurevich et al. (2010) “Laquinimod suppress antigen presentation in relapsing-remitting multiple sclerosis: invitro high-throughput gene expression study” Neuroimmunol. 2010 Apr. 15; 221(1-2):87-94. Epub 2010 Mar. 27.
12. Hla T, Lee M J, Ancellin N, Paik J H, Kluk M J (2001). “Lysophospholipids—receptor revelations”. Science 294 (5548): 1875-8.
13. Hohlfeld et al. (2000) “The neuroprotective effect of inflammation: implications for the therapy of multiple sclerosis”, J Neuroimmunol. 107:161-166.
14. Kleinschmidt-DeMasters et al. (2005) New England Journal of Medicine, 353:369-379.
15. Langer-Gould et al. (2005) New England Journal of Medicine, 353:369-379.
16. McDonald, (2001) “Guidelines from the International Panel on the Diagnosis of Multiple Sclerosis” Ann. Neurol. 50:121-127.
17. Neuhaus et al. (2003) “Immunomodulation in multiple sclerosis: from immunosuppression to neuroprotection”, Trends Pharmacol Sci. 24:131-138.
18. Noseworthy et al. (2000) “Multiple sclerosis”, N Engl J Med. 343:938-952.
19. Paugh S W, Cassidy M P, He H, Milstien S, Sim-Selley L J, Spiegel S, Selley D E (2006). “Sphingosine and its analog, the immunosuppressant 2-amino-2-(2-[4-octylphenyl]ethyl)-1,3-propanediol, interact with the CB1 cannabinoid receptor.”. Mol Pharmacol. 70 (1): 41-50.
20. Paugh S W, Payne S G, Barbour S E, Milstien S, Spiegel S (2003). “The immunosuppressant FTY720 is phosphorylated by sphingosine kinase type 2”. FEBS Lett 554 (1-2): 189-93.
21. Payne S G, Oskeritzian C A, Griffiths R, Subramanian P, Barbour S E, Chalfant C E, Milstien S, Spiegel S. (2007). “The immunosuppressant drug FTY720 inhibits cytosolic phospholipase A2 independently of sphingosine-1-phosphate receptors.”. Blood 109 (3): 1077-85. doi:10.1182/blood-2006-03-011437.
22. Polman et al. (2011) “Diagnostic Criteria for Multiple Sclerosis: 2010 Revisions to the McDonald Criteria”, Ann Neural, 69:292-302.
23. Polman et al., (2005) “Diagnostic criteria for multiple sclerosis: 2005 revisions to the McDonald Criteria”, Annals of Neurology, Volume 58 Issue 6, Pages 840-846.
24. Polman et al., (2005) “Treatment with laquinimod reduces development of active MRI lesions in relapsing MS”, Neurology. 64:987-991.
25. Poser et al. (1983) “New Diagnostic Criteria for Multiple Sclerosis: Guidelines for Research Protocols”, Annals of Neurology, March 1983, 13(3):227-230.
26. Runström et al. (2002) “Laquinimod (ABR-215062) a candidate drug for treatment of Multiple Sclerosis inhibits the development of experimental autoimmune encephalomyelitis in IFN-β knock-out mice”, (Abstract), Medicon Valley Academy, Malmoe, Sweden.
27. Sanchez, T; Estrada-Hernandez, T; Paik, J H; Wu, M T; Venkataraman, K; Brinkmann, V; Claffey, K; Hla, T (2003). “Phosphorylation and action of the immunomodulator FTY720 inhibits vascular endothelial cell growth factor-induced vascular permeability.”. The Journal of biological chemistry 278 (47): 47281-90.
28. Sandberg-Wollheim et al. (2005) “48-week open safety study with high-dose oral laquinimod in patients”, Mult Scler. 11:S154 (Abstract).
29. U.S. Pat. No. 6,077,851, issued Jun. 20, 2000 (Bjork et al).
30. Vollmer et al. (2008) “Glatiramer acetate after induction therapy with mitoxantrone in relapsing multiple sclerosis” Multiple Sclerosis, 00:1-8.
31. Yang et al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.
32. Freireich et al., (1966) “Quantitative comparison to toxicity of anticancer agents in mouse, rat, hamster, dog, monkey and man.” Cancer Chemother Rep 50: 219-244.

What is claimed is:

1. A method of treating a subject afflicted with a form of multiple sclerosis (MS) or presenting a clinically isolated syndrome (CIS) comprising periodically administering to the subject an amount of laquinimod or pharmaceutically acceptable salt thereof, and an amount of dimethyl fumarate (DMF) or pharmaceutically acceptable salt thereof, wherein the amounts when taken together are more effective to treat the subject than when each agent at the same amount is administered alone.

2. The method of claim 1, wherein the laquinimod is laquinimod sodium.

3. The method of claim 1 or 2, wherein the amount of laquinimod is administered via oral administration.

4. The method of any one of claims 1-3, wherein the amount laquinimod is administered daily.

5. The method of any one of claims 1-4, wherein the amount of laquinimod is 0.03-600 mg/day.

6. The method of claim 5, wherein the amount of laquinimod is 0.3 mg/day.

7. The method of claim 5, wherein the amount of laquinimod is 0.6 mg/day.

8. The method of claim 5, wherein the amount of laquinimod is 1.2 mg/day.

9. The method of any one of claims 1-8, wherein the amount of DMF is administered via oral administration.

10. The method of any one of claims 1-9, wherein the amount of DMF is administered daily.

11. The method of any one of claims 1-10, wherein the amount of DMF is 12-7200 mg/day.

12. The method of claim 11, wherein the amount of DMF is 120 mg/day.

13. The method of claim 11, wherein the amount of DMF is 360 mg/day.

14. The method of claim 11, wherein the amount of DMF is 480 mg/day.

15. The method of claim 11, wherein the amount of DMF is 720 mg/day.

16. The method of any one of claims 1-15, wherein the amount of laquinimod or pharmaceutical acceptable salt thereof and the amount of DMF or pharmaceutical acceptable salt thereof when taken together is effective to alleviate a symptom of MS in the subject.

17. The method of claim 16, wherein the symptom is a MRI monitored multiple sclerosis disease activity, relapse rate, accumulation of physical disability, frequency of relapses, frequency of clinical exacerbation, brain atrophy, risk for confirmed progression, or time to confirmed disease progression.

18. The method of any one of claims 1-17, wherein the MS is relapsing MS.

19. The method of claim 17, wherein the accumulation of physical disability is measured by the subject's Kurtzke Expanded Disability Status Scale (EDSS) score.

20. The method of claim 17, wherein the accumulation of physical disability is assessed by the time to confirmed disease progression as measured by Kurtzke Expanded Disability Status Scale (EDSS) score.

21. The method of any one of claims 1-20, wherein the administration of laquinimod substantially precedes the administration of DMF.

22. The method of claim 21, wherein the subject is receiving laquinimod therapy prior to initiating DMF therapy.

23. The method of any one of claims 1-20, wherein the administration of DMF substantially precedes the administration of laquinimod.

24. The method of claim 23, wherein the subject is receiving DMF therapy prior to the initiating laquinimod therapy.

25. The method of any one of claims 1-24, further comprising administration of nonsteroidal anti-inflammatory drugs (NSAIDs), salicylates, slow-acting drugs, gold compounds, hydroxychloroquine, sulfasalazine, combinations of slow acting drugs, corticosteroids, cytotoxic drugs, immunosuppressive drugs and/or antibodies.

26. The method of any one of claims 1-25, wherein the administration of laquinimod or pharmaceutically acceptable salt thereof and DMF or pharmaceutical acceptable salt thereof inhibits a symptom of multiple sclerosis by at least 30%.

27. The method of any one of claims 1-26, wherein either the amount of laquinimod or pharmaceutically acceptable salt thereof when taken alone, and the amount of DMF or pharmaceutical acceptable salt thereof when taken alone, or each such amount when taken alone is not effective to treat the subject.

28. The method of any one of claims 1-27, wherein the subject is a human patient.

29. A package comprising:
a) a first pharmaceutical composition comprising an amount of laquinimod or pharmaceutically acceptable salt thereof and a pharmaceutically acceptable carrier;
b) a second pharmaceutical composition comprising and amount of DMF or pharmaceutical acceptable salt thereof and a pharmaceutically acceptable carrier; and
c) instruction for use for the first and the second pharmaceutical composition together to treat a subject afflicted with MS or presenting a clinically isolated syndrome.

30. The package of claim 29, for use in treating a subject afflicted with MS or presenting a clinically isolated syndrome.

31. Laquinimod or pharmaceutically acceptable salt thereof for use as an add-on therapy of or in combination with DMF or pharmaceutical acceptable salt thereof in treating a subject afflicted with MS or presenting a clinically isolated syndrome.

32. A pharmaceutical composition comprising an amount of laquinimod or pharmaceutically acceptable salt thereof, an amount of DMF or pharmaceutical acceptable salt thereof, and at least one pharmaceutical acceptable carrier.

33. The pharmaceutical composition of claim 32, wherein the laquinimod is laquinimod sodium.

34. The pharmaceutical composition of claim 32 or 33, wherein the amount of laquinimod is 0.03-600 mg.

35. The pharmaceutical composition of claim 34, wherein the amount of laquinimod is 0.3 mg.

36. The pharmaceutical composition of claim 34, wherein the amount of laquinimod is 0.6 mg.

37. The pharmaceutical composition of claim 34, wherein the amount of laquinimod is 1.2 mg.

38. The pharmaceutical composition of any one of claims 32-37, wherein the amount of DMF is 12-7200 mg/day.

39. The pharmaceutical composition of claim 38, wherein the amount of DMF is 120 mg.

40. The pharmaceutical composition of claim 38, wherein the amount of DMF is 240 mg.

41. The pharmaceutical composition of claim 38, wherein the amount of DMF is 480 mg.

42. The pharmaceutical composition of claim 38, wherein the amount of DMF is 720 mg.

43. The pharmaceutical composition of any one of claims 32-42, for use in treating a subject afflicted with MS or presenting a clinically isolated syndrome.

44. Use of:
a) an amount of laquinimod or pharmaceutically acceptable salt thereof; and
b) an amount of DMF or pharmaceutically acceptable salt thereof
in the preparation of a combination for treating a subject afflicted with MS or presenting a clinically isolated syndrome wherein the amount of laquinimod or pharmaceutically acceptable salt thereof and the amount of DMF or pharmaceutically acceptable salt thereof are administered simultaneously or contemporaneously.

45. A pharmaceutical composition comprising an amount of laquinimod for use in treating a subject afflicted with MS or presenting a clinically isolated syndrome, in combination with an amount of DMF, by periodically administering to the subject the pharmaceutical composition and the amount of DMF.

46. A pharmaceutical composition comprising an amount of DMF for use treating a subject afflicted with MS or presenting a clinically isolated syndrome, in combination with an amount of laquinimod, by periodically administering to the subject the pharmaceutical composition and the amount of laquinimod.

47. Laquinimod or pharmaceutically acceptable salt thereof and DMF or pharmaceutically acceptable salt thereof for the treatment of a subject afflicted with MS or presenting a clinically isolated syndrome, wherein the laquinimod and the DMF are administered simultaneously, separately or sequentially.

48. A product containing an amount of laquinimod and an amount of DMF for simultaneous, separate or sequential use in treating a subject afflicted with MS or presenting a clinically isolated syndrome.
